(19) United States
(12) Patent Application Publication
(10) Pub. No.: US 2005/0043783 A1
(43) Pub. Date: Feb. 24, 2005

(54) Helical endoluminal stent and related methods

(76) Inventors: James Peter Amis, Encinitas, CA (US); Hoi Sang U, Santa Fe, CA (US); Charles W. Kerber, La Mesa, CA (US); Horst R. Fichtner, Carlsbad, CA (US)

Correspondence Address: Stout, Uxa, Buyan & Mullins, LLP, Suite 300, 4 Venture, Irvine, CA, 92618, US

(21) Appl. No.: 10/864,280

(22) Filed: Jun. 8, 2004

Related U.S. Application Data
(60) Provisional application No. 60/477,439, filed on Jun. 9, 2003.
(60) Provisional application No. 60/496,135, filed on Aug. 19, 2003.

(51) Int. Cl. A61F 2/06

(52) U.S. Cl. 623/1.22

(57) ABSTRACT

A stent is described that is configured to be inserted into a lumen of a patient. The stent includes a coiled body in a generally helical configuration. In certain aspects, the coiled body includes a strip of multiple strands coupled together. In other aspects, the coiled body includes a strip having one or more lumens in the strip. In addition, certain stents have a coiled body formed by a strip of multiple strands in which at least one of the strands includes a lumen. Pharmaceutical or therapeutic agents may be provided in the lumen to provide a therapeutic effect to a patient receiving the stent. Markers may also be provided in the lumen to facilitate placement of the stent in a patient.

### CROSS-REFERENCE TO RELATED APPLICATIONS

This application claims the benefit of priority of U.S. Provisional Application No. 60/477,439, filed Jun. 9, 2003, and of U.S. Provisional Application No. 60/496,135, filed Aug. 19, 2003. The foregoing applications are commonly assigned and the contents of all are expressly incorporated herein by reference.

### BACKGROUND OF THE INVENTION

1. Field of the Invention

The present invention relates to medical devices, and more particularly to stents that are inserted into a lumen of a patient undergoing a medical procedure. More specifically, the present invention relates to a helically configured endoluminal stent that is sufficiently flexible and strong to provide a desired medical benefit to a patient.

2. Description of Related Art

Stents are typically tube-like structures made of metal or plastic that are inserted into a vessel or passage of a patient to keep the lumen of the vessel or passage in an open state, and to reduce closure of the vessel or passage. For example, stents are frequently used to keep blood vessels open in coronary arteries, used in the esophagus for strictures and/or cancers, and used in ureters to maintain drainage from kidneys, among other things.

### SUMMARY OF THE INVENTION

The present invention herein disclosed relates to a helically configured endoluminal stent that is flexible and strong. The stents in accordance with the present invention are effective in maintaining a lumen of a patient in an open configuration. The stents may also be effective as drug delivery tools to provide one or more therapeutic benefits to a patient. The stents may be effective to provide local and/or systemic therapeutic benefits to a patient.

In one embodiment, the stent includes two or more strands coupled together. The strands are coupled together to form a strip. The strip is configured to have a generally helical configuration. One or more of the strands of the strip may include one or more lumens, which may contain pharmaceutical or therapeutic agents and/or radiopaque materials, alone or in combination. The lumens may extend along the length of the strand, or may be shorter than the length of the strand. The stents may include portions that are biodegradable thereby providing an effective delivery vehicle of pharmaceutical agents contained in the lumens.

In another embodiment, the stent includes a generally helical body defined by a coiled strip, which includes a lumen disposed therein. This embodiment may or may not include one or more strands. As discussed above, the lumen can be used to contain pharmaceutical or therapeutic agents and/or radiopaque materials.

Methods of the present invention include forming a strip of material into a stent having a helical configuration. The method may include forming a lumen in the strip. Alternatively, or in addition, the method may include forming the strip by coupling two or more strands together. The helical configuration may be obtained by changing the temperature of the strip. In one embodiment, the strip is heated beyond its glass transition point so that the strip becomes malleable. The strip is then formed into a helix and is then cooled to a temperature less than the glass transition point so that the helical configuration of the strip is preserved.

Any feature or combination of features described herein are included within the scope of the present invention provided that the features included in any such combination are not mutually inconsistent as will be apparent from the context, this specification, and the knowledge of one skilled in the art.

Additional advantages and aspects of the present invention are apparent in the following detailed description and claims.

### BRIEF DESCRIPTION OF THE DRAWINGS

FIG. 1 is an illustration of a perspective view of an endoluminal helical stent including a plurality of strands forming a strip.

FIG. 2 is an illustration of a perspective view of a strip of the stent of FIG. 1 in a straight configuration.

FIG. 3 is a photograph of an endoluminal helical stent similar to the stent of FIG. 1.

FIG. 4 is an illustration of a perspective view of an endoluminal helical stent including a lumen extending through the strip of the stent.

FIG. 5 is an illustration of a perspective view of an endoluminal helical stent having a lumen.

### DETAILED DESCRIPTION OF THE PRESENTLY PREFERRED EMBODIMENTS

The present invention disclosed herein relates to a stent having a generally helical or coiled configuration that is dimensioned to be inserted into a lumen of a patient. For example, the stent disclosed herein may be inserted into a vascular lumen, such as a blood vessel lumen, a urethral lumen, a tracheal lumen, and/or a nasal lumen, among others. The stent is sufficiently flexible to accommodate relatively abrupt changes in shape as the stent is inserted through a lumen. The stent of the present invention may be effective to treat aneurisms that may be present in a blood vessel. Aneurisms often occur at curves in a blood vessel, and may be associated with relatively poor fluid dynamics. Thus, the stents disclosed herein are sufficiently flexible to accommodate changes in the shape of blood vessels which may be associated with aneurisms. The stent is also relatively strong to maintain the lumen of the patient in an open configuration and also to reduce the possibility of breakage as the stent is inserted to a desired position.

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, front, distal, and proximal are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims. The present invention may be utilized in conjunction with various medical procedures that are conventionally used in the art.

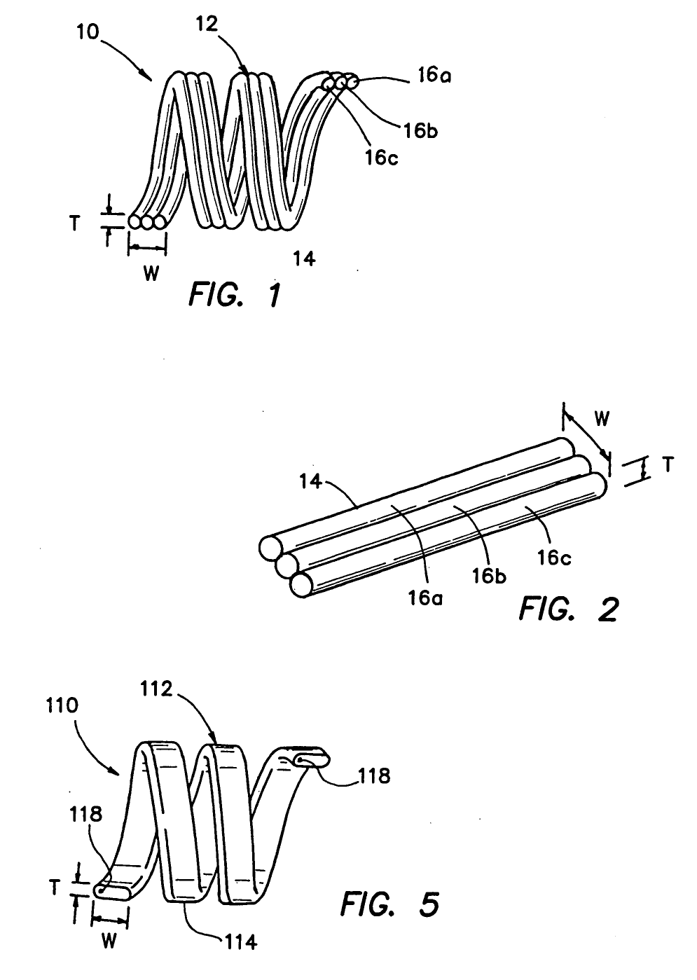

Referring to the figures, and specifically FIG. 1, an endoluminal stent 10 is illustrated including a coiled body 12. The coiled body 12 of the stent of FIG. 1 is defined by a strip 14. The strip 14 includes a plurality of strands 16a, 16b, and 16c. The strands 16a, 16b, and 16c are coupled together to form strip 14. Thus, as can be seen in FIG. 2, the strip 14 has a thickness T which is generally the thickness of a single strand, such as strand 16a, and a width W, which is generally the sum of the diameters of the individual coupled strands. The strip 14, and accordingly, the strands 16a, 16b, and 16c, are formed into a prosthesis. In accordance with one aspect of the present invention, the strands 16a, 16b, and 16c are coiled to form a generally helical configuration. A photograph of stent 10 is illustrated in FIG. 3.

In the embodiment illustrated in FIG. 1, the stent 10 comprises three strands. In other embodiments, the stent 10 may comprise two strands, or may comprise four or more strands. The number of strands that are used to form strip 14 may depend on, among other things, a number of factors, such as the material used to form the strands, the pitch of the helical coils of the stent, the diameter of the stent. The strands 16a, 16b, and 16c are illustrated as each having a generally circular cross-section. In other embodiments of the invention, the cross-sectional shape of one or more of the strands can vary depending on the condition for which the stent is intended to treat.

In the illustrated embodiment, strands 16a, 16b, and 16c are bonded together. For example, the strands may be bonded by use of an adhesive, or by allowing the surfaces of the strands to fuse together during the manufacture of the stent, as discussed herein. By coupling the strands 16a, 16b, and 16c together to form a strip, it is possible to improve the flexibility of the stent relative to stents that have a strip formed of a single piece of material. In addition, by using a plurality of strands compared to a strip formed of only a single strand, the stents disclosed herein can be substantially stronger and/or more flexible than existing stents. The strands 16a, 16b, and 16c are coupled together to form a substantially flat strip, such as strip 14 shown in FIG. 2. The strands 16a, 16b, and 16c are substantially parallel to each other. The strip 14 may then be coiled to form a substantially flat coiled spring-like structure, as shown in FIG. 1, for example.

The stent 10 preferably is formed of a biocompatible material to reduce negative effects, such as antigenic and/or immunogenic responses, associated with the insertion of the stent into a patient. Suitable biocompatible materials are sufficiently flexible and strong to permit the stent to be inserted through a lumen of a patient and to maintain the lumen in an open configuration.

In certain embodiments, portions of the stent 10 may be formed of a biodegradable material, such as a material that degrades over time after the stent is inserted into a patient. In one embodiment, one or more of the strands of strip 14 can be made of a biodegradable material. In another embodiment, a portion of the length of the stent 10 is biodegradable, such as a proximal portion, a distal portion, or a central portion. By forming a portion of the stent 10 of a biodegradable material, relatively less material of the stent will remain in the patient for extended periods of time as, for example, time passes and/or the target site changes (e.g., heals). In addition, in certain embodiments disclosed herein, the biodegradable material may improve the use of the stent as a pharmaceutical or therapeutic delivery device. The stents 10 in accordance with the disclosure herein may include a single biodegradable material, or may include more than one type of biodegradable material. For example, a portion of the stent may be made of a first biodegradable material that degrades at a first rate, and another portion of the stent may be made of a second biodegradable material that degrades at a different rate.

In other embodiments, the strip 14 may be formed of a malleable material, which may be a biodegradable material or a non-biodegradable material. More specifically, the strands of the strip 14 may be formed of a material that has a glass transition temperature or glass transition point greater than a patient's body temperature (e.g., greater than about 37° C). By using such a material, the strip 14 can be heated to above the glass transition point and shaped into a desired configuration, and then cooled in that desired configuration. When the stent 10 is inserted into a patient, the stent will typically generally retain the desired configuration because the patient's body temperature is less than the glass transition point. In addition, the malleable material will have a melting point. The melting point is a temperature, or range of temperatures, where the material transitions from a solid phase to a liquid phase. When coupling the individual strands of strip 14 together, the strands are heated to their approximate melting point so that the surfaces of the strands can fuse and bond to each other to form a strip when they cool.

In certain embodiments of the invention, all or portions of the stent may be made of a resorbable polymeric material, such as the materials disclosed in co-pending and commonly assigned U.S. application Ser. No. 10/375,451, filed Feb. 27, 2003 or U.S. Provisional Application No. 60/408,393, filed Sep. 4, 2002, both of which are hereby incorporated by reference in their entireties. For example, the stents of the present invention may include polymers, including copolymers, of lactide, such as L-lactide, D-lactide, D,L-lactide, and combinations thereof. Alternatively, or in addition, the stents may include polymers of glycolide, trimethylene carboneate, caprolactone, and/or combinations thereof. In certain embodiments, the stents include a polylactide, such as a copolymer of L-lactide and D,L-lactide. More specifically, one embodiment of a stent includes a copolymer that comprises about 60-80% of L-lactide, and about 20-40% of D,L-lactide. In a particular embodiment, the stent comprises poly (L-lactide-co-D,L-lactide) 70:30 Resomer LR708 manufactured and supplied from Boehringer Ingelheim KG of Germany. In other embodiments, the stent comprises polymers derived from one or more cyclic esters, such as lactide (i.e., L, D, D,L, or combinations thereof), epsilon-caprolactone, and glycolide. In one such embodiment, the stent comprises about 1-99% epsilon-caprolactone, in another embodiment, the stent comprises about 20-40% epsilon caprolactone. A further stent may comprise 65:35 poly (L-lactide-co-epsilon-caprolactone). Another stent comprises 90% poly (L-lactide-co-D,L-lactide) (PLLA) 70:30 and 10% capralactone.

As illustrated in FIG. 4, one or more of the strands 16a, 16b, and 16c may include a lumen 18 disposed in the strand. In the embodiment shown in FIG. 4, strand 16a has a lumen 18 extending along the length of the strand. The lumen 18 is illustrated as extending from one end of strand 16a to the other end of strand 16a.

In modified embodiments, lumen 18 may be provided in multiple portions (such as proximal and distal portions) and/or orientations of one or more strands such that a length of one or more of the lumens is less than the length of the corresponding strand or strands. Alternatively, or in addition, one or more lumens may be provided in the strip that are oriented in a non-parallel configuration to the length of the strip. For example, in modified embodiments lumens may be provided at angles of about zero to about ninety degrees relative to the length of the strip such that the lumen or lumens transverse at least a part of the strip. In certain embodiments, the lumens may be provided at an orthogonal orientation with respect to the length of the strip.

Transversing lumens, defined as diagonal lumens (e.g., lumens that are neither parallel nor orthogonal to the length of the strip) or orthogonal lumens, may transverse two or more strands of the strip. The transversing lumens can accordingly provide communication paths or substance delivery structures, between strands of the strip. However, the transversing lumens do not necessarily have to transverse multiple strands of the strip, and each traversing lumen can instead traverse only a single strand and terminate in proximity to the junction between two strands so that such traversing lumens do not extend between the strands, but provide incremental lumen portions in individual strands of the strip. In one embodiment, an orthogonal or non-parallel exit lumen of a strand can intersect and fluidly communicate with a parallel-oriented lumen of the strand so that the parallel-oriented lumen has an exit path to an outer surface of the strand, and the parallel-oriented lumen may terminate at the exit or continue along a length of the strand. Moreover, exit lumens may be positioned at different positions along lengths of different strands, and/or other exit lumens may fluidly connect parallel-oriented lumens of two or more adjacent strands. The fluid connections may form isolated or sealed flow paths between the two or more parallel-oriented lumens or may fluidly connect the two or more parallel-oriented lumens not only to one another but also to an outer surface of the stent. Exit lumens can be disposed on inner surfaces and/or on outer surfaces of each strand.

In certain embodiments, the lumen 18 is configured to contain one or more pharmaceutical or therapeutic agents. Pharmaceutical or therapeutic agents that may be provided in the lumen 18 include pharmaceuticals and therapeutics, such as pain medications, chemotherapeutic agents, anti-stenosis agents, proteins, vectors for gene therapy, and/or cells, such as stem cells, among other things. For example, pharmaceutical or therapeutic agents include, but are not limited to, analgesics, anesthetics, such as local anesthetics, antibiotics, steroids, anti-tumor agents, hormones, and hormone-like agents. Examples of antibiotics include, but are not limited to, ampicillin, chloramphenicol, chlotetracycline, clindamycin, erthromycin, gramicidin, gentamicin, mupiroicin, neomycin, polymyxin B, bacitracin, silver sulfadiazine, tetracycline, and chlortetracycline. Examples of hormones and other related drugs useful with the present invention include, but are not limited to human growth hormone (HGH), bone morphogenic proteins (BMPs), transforming growth factors (TGF-β), interferons, interleukins, calcitonin, estrogen, and 17-β-estradiols. Examples of anti-inflammatory drugs include, but are not limited to, cortisone, Nonsteroidal Anti-Inflammatory Drugs (NSAIDs), and interleukin 3 inhibitors. Examples of anti-tumor agents include cytotoxic materials that are effective in targeting and killing malignant cells more selectively than non-malignant cells. Examples of anti-stenosis agents include cell proliferation inhibitors, such as inhibitors of smooth muscle cell proliferation. Some anti-stenosis agents include Rapamycin commercially available from the Wyeth Ayerst company (Sirolimus®), and Paclitaxel commercially available from the Bristol-Myers Squibb Company (Taxol®). Other anti-stenosis agents include heparin, other taxanes, tacrolimus, actinomycin D, angiopeptin, vassenoids, flavoperidol, estrogen, halofuginone, matrix metallopreteinase inhibitors, ribosimes, interferons, and antisense compounds.

When pharmaceutical or therapeutic agents are provided in the lumen 18, the stent is configured to permit the pharmaceutical or therapeutic agents to be released from the lumen 18 when the stent is inserted into a patient. The release of the pharmaceutical or therapeutic agents can be controlled by a person or machine, or can occur passively. In addition, the pharmaceutical or therapeutic agents can be released for extended periods of time or as a single bolus. When the pharmaceutical or therapeutic agents are released over extended periods of time, the release may be periodic such that desired amounts of the agents are released in a given period of time. For example, when the stent 10 includes a biodegradable strand, a lumen 18 may be provided in the biodegradable strand. A pharmaceutical or therapeutic agent is provided in the lumen of the biodegradable strand. As the biodegradable strand degrades in the patient, the pharmaceutical or therapeutic agent is released to provide a desired therapeutic effect, including pain relief.

In additional embodiments, the lumen 18 may be provided with a radiopaque material that is useful to facilitate radiographic positioning of the stent 10 within a patient. For example, a carbon fiber may be inserted into the lumen 18 to facilitate radiographic visualization of the stent when the stent is in the patient. One advantage of using a carbon fiber located in a lumen is that it may provide effective visualization while minimizing distortion or obliteration of the image of the stent. Thus, the stent 10 may comprise a radiopaque marker. The radiopaque marker may be located on the stent or may be located in the stent, such as by being located in a lumen of the stent. In one embodiment, the radiopaque marker may include a carbon fiber integrally formed within or along an outer surface of at least one of the strands of strip 14.

The lumens of the stents of the present invention may be sealed to prevent unintentional release of pharmaceutical agents and/or radiopaque materials. For example, the lumens may be sealed by closing the ends of the lumens by bonding the ends of the strip or strands together, such as by melting the ends of the strip or strands. Alternatively, a membrane may be provided on the ends of the lumens. The membranes may be biodegradable, so that the pharmaceutical agents may be released only after the stent is inserted in a patient. Or, the membranes may be permeable, so that the pharmaceutical agents can diffuse through the membrane when the stent is inserted in a patient.

In certain embodiments, the stents may include a plug located in the lumen 18. Plugs may be effective in preventing the pharmaceutical or therapeutic agents from being released from the lumen 18 before the stent is placed in a patient, and in facilitating the release of the pharmaceutical or therapeutic agents after the plug is no longer effective to seal the lumen 18. Depending on the configuration of the stent and the lumens located in the stent, one or more plugs may be used in the stent. For example, a first plug may be provided in a lumen at a proximal end of the stent, and a second plug may be provided in a lumen at the distal end of the stent. Plugs that are located at the ends of the stent may include a first region that is sized to be placed in the lumen of the stent, and a second region that has a diameter greater than the lumen of the stent. In certain embodiments, the plugs may have a second region that has a diameter greater than the thickness of the stent. In addition, a stent may include a plurality of plugs disposed in the lumen. For example, one or more plugs may be provided in the lumen spaced away from an end of the stent, and in addition to or alternatively one or more of the same sized plugs may be disposed at one or both of the ends of the stent. Thus, the plugs may be effective to create one or more compartments within the lumen.

Plugs that are used in conjunction with stents having a lumen are preferably manufactured from a biocompatible material. Plugs may include a biodegradable material such that the plug degrades over time after the stent is placed in a patient. Suitable biodegradable materials include those biodegradable materials identified above with respect to the stent. For example, the stent may include a first plug formed of a biodegradable material that degrades at a first rate, and a second plug formed of a biodegradable material that degrades at a second rate.

The plugs used with the stents disclosed herein may be configured to release contents stored in the lumen 18 by a number of different methods. For example, when biodegradable plugs are placed in the lumen 18, the plug may dissolve or degrade and allow the pharmaceutical or therapeutic agents to pass from the lumen as the plugs dissolve or degrade. The plug may become dislodged from the stent as it begins to dissolve which may facilitate a more rapid release of the pharmaceutical or therapeutic agents contained in the lumen 18. In addition, the plug may be displaced from the lumen by the blood flowing pass the stent. Thus, such a plug would typically be placed in the lumen of the stent so that it engages the body of the stent to prevent leakage of the pharmaceutical or therapeutic agents contained in the lumen. When the stent is placed in a patient, the blood flow around the stent acts on the plug to cause it to be displaced from the lumen so that the pharmaceutical or therapeutic agents may be released. In certain embodiments, the blood flow pushes the plug from the lumen. In other embodiments, the blood flow pulls the plug from the lumen.

As an alternative to the placement of a plug into a lumen, an end or intermediate portion of a lumen may be heat-clamped shut wherein heat and optional pressure are applied to deform and at least partially occlude a portion of a lumen shut. Moreover, a resorbable material may be brought to a gel or liquid phase, applied to an end of a lumen to occlude the lumen, and then allowed to cool and harden. Any of the above-described plugs or occluding mechanisms may be positioned within one or more lumens of the stent.

In modified embodiments, other portions or all of the stent may be coated with any of the compositions or agents disclosed herein, wherein the stent before coating may be non-resorbable, partially resorbable, or entirely resorbable. According to one such embodiment, outer surfaces of the stent that contact a vessel wall, but not inner surfaces, may be coated. For example, a stent in accordance with the disclosure herein, may include a portion, strip or surface thereof that comprises an anti-stenosis agent. The anti-stenosis agent may be coated on the strip, or may be provided in a lumen of the strip, or may be used in forming the strip. When an anti-stenosis agent is used in forming a strip, it may be desirable to utilize a matrix which comprises the anti-stenosis agent.

Stents that include a plurality of strands, such as stent 10, may be configured to provide greater friction when placed in a vessel relative to a stent which comprises only a single strand. Advantageously, the improved friction obtained with the present stents may facilitate proper fixation of the stent in a vessel lumen near a target site, and may help enhance delivery of pharmaceutical or therapeutic agents near the target site. In additional embodiments, including single stranded embodiments discussed herein, a stent may include one or more projection members extending from the stent. The projection members are typically effective to engage the stent with a wall of the vessel in which the stent is inserted. Examples of projection members include cleats, ribs, spikes, and hooks or barbs. Projection members may be integrally formed with the stent or may be attached or affixed to the stent. Alternatively, or in addition, a stent may comprise a strip with an uneven surface topography, such as a roughened or irregular surface, which may be effective to enhance the engagement of the stent with the vessel wall. Protuberances or other surface deviations or variations such as set forth in U.S. Pat. No. 6,391,059, the entire contents of which are incorporated herein by reference, may also be formed on one or more surfaces or portions of surfaces of the stent.

As indicated herein, the endoluminal helical stent disclosed herein is configured to be inserted into a vascular lumen, a urethral lumen, a tracheal lumen, a nasal lumen, and other lumens of a patient. In certain instances, lumens in which the stent is to be inserted have outer diameters ranging from about 2 mm to about 10 mm. As one example, and not by way of limitation, the strip of the stent may have a thickness of about 0.75 mm, and a width of about 2.5 mm. The strands of such a strip may have diameters of about 0.75 mm. The strip of the stent may have a pitch in a range from about 2.5 mm to about 10 mm, and in certain embodiments, the pitch can be about 6.0 mm. In one example, the length of the strip may be between about 25 mm and about 30 mm when the strip is formed into a helically coiled body. When a lumen is provided in one or more of the exemplary strands, the lumen may have a diameter in a range from about 20 micrometers to about 200 micrometers.

By coiling the strip 14 of stent 10 to form body 12, the stent acquires an elastic property which enables the stent to assume a plurality of configurations. For example, the strip 14 of the stent 10 may be deformable so that the stent can be arranged in an active first configuration, such as when the stent is being inserted through a lumen of a patient, and so that the stent can assume a relaxed second configuration when the target site is reached. In other words, the helical strip 14 may be deformable between an active first configuration and a relaxed second configuration, such that when the strip is placed into the first configuration the strip has a bias to return to or assume the second configuration. Certain stents may be self-expanding when they are placed in a vessel. In certain embodiments, the helical configuration of the stent is determined, at least in part, based on the curvature of the vessel in which the stent is to be inserted. Some stents may include a left handed helical configuration, and other stents may include a right handed helical configuration. By providing vessel-specific helical configurations, the stent can be effectively placed within a vessel and provide an effective engagement with the vessel walls.

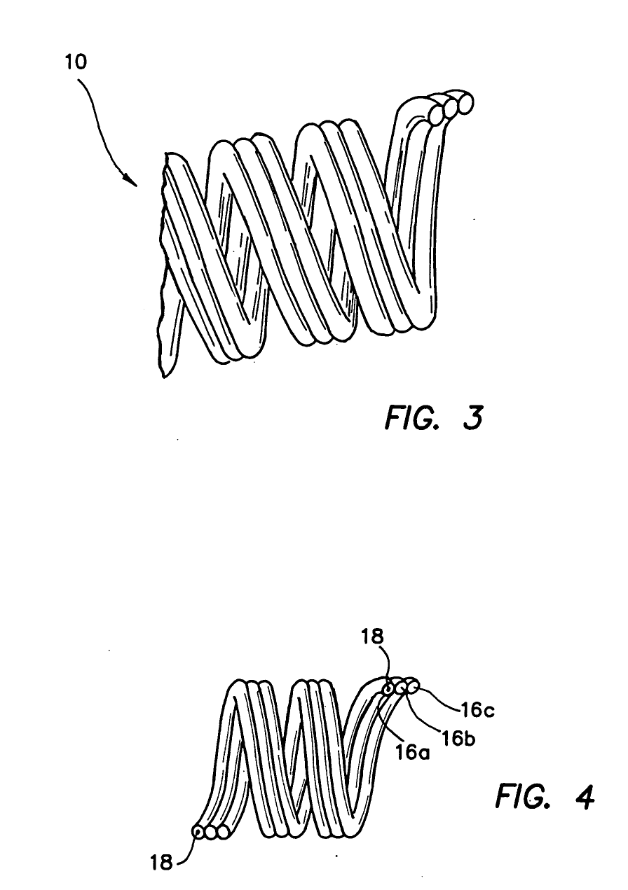

In another embodiment of the invention, a stent comprises a generally helical body defined by a generally flat coiled strip. The coiled strip in this embodiment can include at least one lumen in the strip. One difference between this embodiment, and the embodiment described above is that the strip does not necessarily include two or more strands, such as strands 16a, 16b, and 16c of FIGS. 1-4. An example of this embodiment of the stent is illustrated in FIG. 5, wherein like parts are identified by like reference numbers increased by 100. Thus, in reference to FIG. 5, a stent 1 10 comprises a coiled body 112. Coiled body 112 is defined by strip 114, which has a lumen 118 extending through the strip 114. Although comprising a single coiled strip (e.g., a flat coiled strip) instead of multiple coiled strands, the stent 110 may otherwise comprise one or more of the features and configurations set forth above. A few exemplary embodiments and implementations of the stent 110 are set forth below.

Similar to the strip 14 of FIGS. 1-4, strip 114 has a thickness T′ and a width W′. Generally, and as illustrated, the width W′ is greater than the thickness T′. In the illustrated embodiment, strip 114 has a generally rectangular cross-section. In additional embodiments, strip 114 can have other geometrically-shaped cross sections.

The lumen 118 is illustrated as extending from a first end to a second end of the strip 114. In other embodiments, the strip 114 may have a lumen that is shorter than the length of the strip. In further embodiments, the strip 114 may have a plurality of lumens in the strip. By providing at least one lumen in the strip 114 of the stent 110, the flexibility and/or strength of the stent can be controlled and predetermined depending on the particular type of medical procedure. Similar to the embodiment of FIGS. 1-4, the lumen 118 may act as a pharmaceutical or therapeutic agent delivery tool. For example, one or more pharmaceutical or therapeutic agents may be provided in the lumen 118 which can be selectively released from the stent after the stent is placed in a patient, as discussed above. One way of controlling the release of a pharmaceutical or therapeutic agent from the stent 110 is to form portions of the strip 114 from a biodegradable material, as discussed above. In certain embodiments, a proximal portion, a distal portion, and/or a central portion of the strip 114 may comprise a biodegradable material. In other embodiments, a lateral portion of the strip 114, for example, a portion that extends along the length of the strip 114, but not the entire width of the strip 114 may be made of a biodegradable material. When more than one portion of the stent 110 includes a biodegradable material, it may be desirable to form the different portions, such as a first portion and a second portion, of biodegradable materials that degrade at different rates, as discussed herein.

In addition, and similar to the lumen 18 as discussed herein with respect to stent 10, the lumen 118 may include a radiopaque material, which may be useful to facilitate identification of the stent 110 when it is inserted in a patient. One example of a radiopaque material includes a carbon fiber located in the lumen. Alternatively, or in addition, one or more radiopaque markers may be located on or within the strip 110 as compared to being provided in a lumen of the strip 114.

Although the stent 110 is illustrated without a plurality of strands, strands may be provided at least along portions of the strip 114. The strands in such embodiments are arranged in a substantially parallel configuration, and they may be bonded together, as discussed herein. In other embodiments, the strandless strip 114 may be machined or molded to include regions devoid of materials to simulate a plurality of strands. Such regions are similar to the regions between strands 16a and 16b, and 16b and 16c of the stent 10 of FIGS. 1-4. In modified embodiments, one or more strands or regions of the stent 110 may be formed (e.g., machined) to have longitudinal axes at angles from about zero to about 90 degrees relative to a longitudinal axis of the stent 110.

The stents disclosed herein are typically formed of a material and a shape that are effective to permit the stent to deform to different widths and shapes in a vessel lumen when the stent is in an expanded or relaxed configuration, as discussed above. For example, a stent may comprise a first portion having a first diameter, and a second portion having a second diameter, where the second diameter is different than the first diameter.

The stents in accordance with the present invention may be made using any conventional method known by persons of ordinary skill in the art. In one embodiment, the stents are formed by an extrusion process. The strips 14 or 114 may be extruded as a single structure using, for example, a single extrusion output orifice matching a cross-sectional shape of the stent 110, and coiled around a mandrel, or other similar tool, to form a helix or coiled structure. Alternatively, individual strands, such as the strands 16a, 16b, and 16c may be individually extruded using a separate extrusion output orifice for each strand.

When the strip is manufactured according to the latter implementation of an extrusion process, a plurality of strands, exiting their respective output orifices at their melting points, can be placed/bonded together and cooled so that the strands are coupled to form a strip. The individual strands can be arranged in a helical configuration before they are bonded or during bonding, or they can be arranged in a helical configuration after they have been bonded to form a strip, such as the strip 14. Thus, in accordance with the present invention, a method of making an endoluminal stent, such as the stent 10, includes coupling a plurality of strands to each other in a generally parallel configuration to form a strip, and forming the strip into a helix dimensioned to be inserted into a lumen of a patient. As discussed herein, the coupling can include physically bonding a first strand to a second strand.

In one embodiment, the method optionally includes one or more steps of causing the temperature of the strands to be between a glass transition point and a melting point, such as by heating the strands to a desired temperature; wrapping the strands around a mandrel as the strands are removed from an extruder; and causing the temperature of the strands to be less than the glass transition point so that the strands bond to each other, such as by cooling the strands.

Any of the above methods or other method may include forming at least one lumen in the strand, or in the strip, of the stent. The lumen can be formed using any effective technique, such as machining, which would be recognized by persons skilled in the art as suitable upon a reading of the present disclosure. The strip or at least one of the strands may be formed with, for example, a centrally-disposed lumen, using for example an air-blowing technique wherein pressurized air is directed through a center cross-sectional area of the strip or strand as the strip or strand is extruded. Methods of the invention can also include providing a pharmaceutical agent in the lumen, as discussed herein. The pharmaceutical agents can be infused into the lumen, such as by using a needle and/or other similar instruments, or coated on an interior surface of the lumen. The pharmaceutical agents can be provided in the lumen at the time the stent is manufactured, or at a time of the surgical procedure. Similarly, methods of the invention can include providing a carbon fiber, or other radiopaque material, in the lumen of the strip. The carbon fiber, or other radiopaque material, can be inserted through a lumen after the lumen is formed in the strip, or can be integrally formed in the strip.

In additional embodiments of the invention, helically configured stents may include one or more portions having a coil with a first pitch, and one or more other portions having a coil with a second pitch, wherein the first pitch and the second pitch are different. For example, a first portion of such a stent may include coils with a small pitch, and a second portion of the stent may include coils with a large pitch. The portions may be near the ends of the stent, or may be in the central region of the stent. Varying the pitch of the helical stent permits different stents to be manufactured that are appropriate for different medical conditions for which they are being used.

In other embodiments of the invention, which may or may not be combined with the above embodiments, a helically configured stent may include a portion having a first stent diameter and first physical characteristics as set forth herein, and one or more other portions of the stent may have a second stent diameter and second physical characteristics (selected from for example those described herein), wherein at least some of the first diameter and first physical characteristics, and the second diameter and second physical characteristics, are different.

In further embodiments of the invention, which may or may not be combined with the above embodiments, helically configured stents may include one or more portions having a first cross-sectional shape and/or area, and one or more other portions having a second cross-sectional shape and/or area, wherein the first cross-sectional shape and/or area and the second cross-sectional shape and/or area are different. For example, a first portion of such a stent may include three strands with a relatively small pitch and a relatively small cross-sectional area, and a second portion of the stent may include coils with a larger pitch and a larger cross-sectional area. The portions with the smaller dimensions may be near the ends of the stent in one implementation, or may be in a central region of the stent in other implementations.

In additional embodiments of the invention, the strips of the stents may have one or more lumens that contain one or more pharmaceutical or therapeutic agents, as discussed herein. The thickness of the strip material between the lumen and the outer surface of the strip may be irregular along the length of the strip. For example, the strips may be provided with indentations that form pores into the lumen over time. Alternatively, the strips may be provided with pores on outer surfaces of the strip, such that pharmaceutical agents can diffuse through the pores from the lumen when the stent is inserted in a lumen of a patient. Additional configurations of the stent that are suitable for controlling delivery of pharmaceutical or therapeutic agents are disclosed in co-pending and commonly assigned U.S. application Ser. No. 10/129,214, filed Oct. 21, 2002, which is hereby incorporated by reference in its entirety. By employing the features disclosed for example in U.S. application Ser. No. 10/129,214, the delivery of the pharmaceutical or therapeutic agents from the stents disclosed herein may be controlled and/or monitored. The stents can accordingly be similarly used to govern growth of soft and/or hard tissues, in addition to maintaining lumens in open configurations.

The stents in accordance with the present invention may be inserted into a lumen of a patient using any suitable technique known to persons skilled in the art. For example, the stent may be provided in a sleeve that constricts the stent so that the stent can be inserted through a lumen of a patient. Once the stent is in a desired position, the sleeve can be retracted from the stent so that the stent can expand and assume a second relaxed configuration, which is effective to maintain the lumen of the patient in an open configuration.

In a particular embodiment, a stent having a helical configuration is coupled to a guidewire, which may be radiopaque. Typically, the guidewire is coupled to the stent at the guidewire's distal end. The guidewire may be coupled to the stent by bonding, such as by use of an adhesive or the like. Or, the guidewire may be welded to the stent, for example by heating the stent to its melting temperature and allowing it to cool when the guidewire is contacting the melted portion of the stent. The guidewire may pass along the exterior of the strip of the stent, or it may be placed within a lumen of the stent, such as lumen 18 or 118, described herein. In some embodiments, the stent may be formed with a single lumen. The guidewire may be used with a stent in either the stent's helical or non-helical configurations. In certain embodiments, as discussed herein, the guidewire may provide a substrate that is effective to maintain the stent in a non-helical configuration, such as an elongated, relatively straight, configuration.

The combination of the stent and the guidewire may then be inserted into a lumen of a vessel of a patient. Typically, the stent and guidewire will be advanced within the lumen to a target site where the stent is needed. The target site may be an aneurysm in a blood vessel, such as a small mouth aneurysm or a large mouth aneurysm. Or, the target site may be an area where a pharmaceutical or therapeutic agent is needed to provide a therapeutic effect. For example, using a stent which includes a chemotherapeutic agent, it is possible to provide a local chemotherapeutic treatment near a tumor. Or, the target site may include a vessel region that is narrowed or constricted.

In one embodiment, the helical stent is urged into a non-helical configuration before it is inserted into the lumen of the patient. For example, the stent may be elongated or straightened from its helical configuration so that the effective cross-sectional diameter of the stent is reduced during the insertion step. As discussed herein, when the stent is released at its target site, it can then expand into its helical configuration to provide the desired therapeutic effect. By utilizing compositions as disclosed above, such as a softening agent, e.g., capralactone, in combination with a resorbable material, e.g., PLLA, in the manufacture of the stent, the present stents can be inserted to a target site with reduced damage relative to stents made of only a resorbable composition, such as only PLLA. For example, stents which have no or relatively little amounts of softening agents may break, kink, or otherwise become damaged when the stents' shape changes. In comparison, stents disclosed herein, which include a softening agent, such as capralactone, are able to accommodate reshaping with little or no damage. Generally, as the amount of softening agent increases in the stent, the amount of damage to the stent during reshaping decreases. Indeed, the stents disclosed herein are able to be reshaped, for example, by reshaping a coiled stent into a straightened stent, at room temperature (e.g., about 20 degrees celsius), without being damaged and/or permanently deformed. In addition, the methods of inserting the stent herein may avoid trauma and damage that may be associated with conventional balloon stent procedures.

When the stent is in proximity to the target site, the guidewire may be uncoupled from the stent. The guidewire may be uncoupled by heating the bond between the guidewire and the stent. In certain embodiment, the guidewire is heated, and preferably, the distal end of the guidewire is heated. The bond between the guidewire and the stent may be heated by a number of different methods. For example, a heating element may be placed in proximity to the distal end of the guide wire to provide local heating of the bond. The heating element may be an integral component of the endoluminal system disclosed herein, or may be a separate element that is inserted in a patient. Or, the guidewire may be heated by passing electrical current through the guidewire. In embodiments where substantial portions of the guidewire are heated, regions of the guidewire located away from the distal end may be insulated to provide more localized temperature changes and control. In a preferred embodiment, only a distal end or tip of the guidewire is heated. In addition, a cooling device may be provided around regions of the guidewire to help control the heating of portions of the guidewire.

Typically, an amount of heat is generated near the guidewire, such as near the distal end of the guidewire, to reshape the stent. For example, the distal end may be heated to a temperature approaching or mildly exceeding the glass transition temperature of the stent so that the stent (e.g., within a fluid filled vessel) can change from an uncoiled configuration, which was used during the insertion step, to a coiled configuration, as the heated part of guidewire is moved along the length of the stent to be coiled. In one embodiment, a heated tip of a guidewire is moved proximally from a distal end of a straigntened stent proximally causing the stent to be heated and to coil as the heated guidewire part is moved proximally and out of (i.e., removed from) the distal portion of the stent. In another embodiment, a guidewiere or other heating device is not disposed within a lumen of the stent and is moved distally relative to the stent (e.g., the stent may be retracted relative to the heating device) to heat and coil the stent within for example a vessel.

For the stents disclosed herein, the glass transition temperature may range from between about 100 degrees Farenheit to about 120 degrees Farenheit. Stents made of other materials may have higher or lower glass transition temperatures. In one embodiment, the stent and guidewire may be inserted towards a target site in a catheter or other hollow device. To facilitate the reshaping of the stent from an uncoiled configuration to a coiled configuration, the distal end of the guidewire may be heated, as discussed herein. When the guidewire has been heated to the desired temperatue, the catheter and guidewire may be retracted while the stent remains in place near the target site, as discussed herein. Alternatively, when the guidewire has been heated to the desired temperature, the stent may be distally advanced from the catheter past the guidewire where the stent assumes the coiled configuration. In embodiments in which the guidewire is located in a lumen of the stent, the guidewire may provide structural rigidity to facilitate shaping the stent into an uncoiled or non-helical configuration, to facilitate insertion of the stent through the vessel lumen. As the distal end of the guidewire is heated, and as the stent passes from the guidewire distal end, the stent naturally assumes a coiled or helical configuration being heated to its glass transition temperature and forming back to it's pre-set shape. The amount of heat may be varied during the reshaping procedure to alter the particular configuration of the coiled helical stent. For example, the guidewire may be heated periodically as the guidewire is removed from the stent to create regions of the stent that have different pitches or other different properties obtained from varying the temperature of the guidewire or heating element. In accordance with the disclosure herein, the stents have an active configuration and a relaxed configuration. Thus, when the stent passes from the guidewire, and it is heated, the stent naturally attempts to assume its relaxed, helical configuration as it cools from its glass transition temperature.

After the guidewire is uncoupled from the stent, the stent expands, as described herein, to obtain its relaxed configuration. The guidewire may then be removed from the patient. The guidewire may be removed while it is being heated.

The stents disclosed herein are especially useful in situations where multiple stents may be desired, or it may be desired to re-stent a target site. With currently available stents, it is not possible to re-stent a vessel, let alone insert multiple stents in proximity to each other. Thus, a method may include an additional step of inserting a second stent in proximity to the first stent. For example, the second stent may have a smaller diameter than the first stent, and may be inserted within the helix of the first stent. When the first stent is made of a biodegradable material, the second stent may be inserted in proximity to the first stent after some or all of the first stent has degraded.

As the stent approaches the target site, a method may include a step of engaging the stent with a vessel wall or an occluded area, such as partially occluded or fully occluded areas, in the vessel. In one embodiment, the engaging step may include a step of rotating the stent to cause the lumen of the vessel to enlarge as the stent is rotated. In situations where the stent includes a right-hand thread, the stent may be rotated and/or advanced by rotating the stent to the right which may accommodate advancement of the stent through certain curves or other features of a vessel. In situations where the stent includes a left-hand thread, the stent may be rotated and/or advanced by rotating the stent to the left which may facilitate for example maneuvering around or into for example a left hand turn of a vessel. Rotation of the stent can also advance the stent into a smaller diameter portion of a vessel. Thus, rotation of the stent can facilitate advancement of the stent and/or enlargement of the vessel lumen and/or occluded area. In one embodiment, the stent may have a distal end with a smaller diameter compared to a proximal end or region. For example, the stent may have a distal end with a diameter of “X” and a region that is proximal, such as a central region, or the proximal end of the stent, that has a diameter greater than “X”. Thus, as the stent is rotated and advanced into the vessel or obstruction, the relatively wider portion can facilitate enlargement and opening of the vessel and/or obstacle. As discussed herein, a stent that includes a plurality of strands coupled together may be particularly advantageous due to the friction created between the veseel and/or obstacle and the varying surface topography of the stent.

Alternatively, or in addition, such a stent may include a protuberance, such as a rib, barb or hook, which permits the stent to engage with the wall and allows the stent to maintain a desired configuration as the guidewire is removed. In a further embodiment, the stent is constructed so that once the distal end is fixedly engaged with the vessel or the obstacle, the direction of rotation can be reversed to increase the diameter of the fixed stent. In other words, regions of the stent proximal to the distal end may be rotated in an opposite direction relative to the initial direction the stent was rotated when it was advanced (whereby the distal end remains in fixed engagement to the vessel or obstacle) causing a proximal region to expand and increase in cross-sectional size. For example, a stent with one or more protuberances, or other engagement means for frictionally engaging a vessel wall or obstruction, disposed only on the distal end of the stent may be rotated and/or advanced to a target site in a vessel. The protuberances may then engage the vessel wall to fix the distal end of the stent in the vessel. Once the distal end of the stent is fixed in position, the stent may be rotated in the opposite direction (e.g., counter-rotation) relative to the direction that was used to insert the stent. Because the distal end of the stent is fixed by the frictional engagement of the protuberances and the vessel wall or obstruction, the distal end does not rotate substantially by the counter-rotation applied to the stent. Due to the coiled shape of the stent, the counter-rotation of the stent causes the proximal portions of the stent (proximal to the fixed distal end) to expand and increase in diameter. In one embodiment, a portion of the stent may be heated as it is counter-rotated to facilitate reshaping of the stent shape. Once the desired dimensions are obtained, the heat and/or the counter-rotation may be stopped. The stent is able to maintain the expanded configuration due, at least in part, to the materials that the stent is manufactured from.

In accordance with the present invention, a stent may be inserted into a patient by twisting the coiled body of the stent into a first configuration having a smaller diameter than when the coiled body is in a relaxed configuration. For example, the stent may be twisted to reduce the diameter of the coiled body by twisting the strip of the stent around a catheter device, or other similar object. The catheter device with the stent may then be inserted into a lumen of a patient. When the stent is at a desired location, the catheter can be removed, and the stent can assume its relaxed configuration to maintain the lumen of the patient in an open configuration.

While this invention has been described with respect to various specific examples and embodiments, it is to be understood that the invention is not limited thereto and that it can be variously practiced within the scope of the following claims. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

1. An endoluminal stent, comprising a plurality of strands coupled together to form a strip arranged in a generally helical configuration.

2. The stent of claim 1, comprising three strands.

3. The stent of claim 1, wherein at least one of the strands includes a biodegradable material.

4. The stent of claim 1, wherein at least one of the strands includes a material having a glass transition temperature greater than a patient's body temperature.

5. The stent of claim 1, wherein the plurality of strands are coupled together and are effective to provide enhanced flexibility to the stent, relative to stents consisting of a single strand and having an equal strength and an equal diameter and pitch.

6. The stent of claim 1, wherein the plurality of strands are coupled together and are effective to provide enhanced strength to the stent relative to stents consisting of only one strand and having an equal strength and an equal diameter and pitch.

7. The stent of claim 1, wherein at least one of the strands includes a lumen disposed in the strand.

8. The stent of claim 1, wherein the strip comprises a biodegradable strand and a non-biodegradable strand, the biodegradable strand including a lumen configured to contain a pharmaceutical agent effective to provide a therapeutic effect as the biodegradable strand degrades.

9. The stent of claim 1, further comprising a radiopaque marker located on the stent and being effective to provide positional information of the stent as it is placed in a lumen of a patient.

10. The stent of claim 1, wherein at least one of the strands includes a radiopaque material.

11. The stent of claim 10, wherein the radiopaque material includes a carbon fiber.

12. The stent of claim 1, further comprising a carbon fiber integrally formed along at least one of the strands.

13. The stent of claim 1, wherein the plurality of strands includes a biodegradable first strand, and a biodegradable second strand, the first strand degrading at a different rate than the second strand.

14. The stent of claim 1, wherein the strands have a diameter of approximately 0.75 mm.

15. The stent of claim 1, wherein the strip has a width of about 2.5 mm and a thickness of about 0.75 mm.

16. The stent of claim 1, wherein at least one of the strands has a lumen with a diameter between about 20 micrometers and about 200 micrometers.

17. The stent of claim 1, wherein the strip has a pitch having a range of about 2.5 mm to about 10 mm.

18. The stent of claim 1, wherein the strip has a pitch of about 6.0 mm.

19. The stent of claim 1, wherein the helical strip has a length between about 25 mm and about 30 mm.

20. The stent of claim 1, wherein the strip is arranged in a helix dimensioned to be placed in a vascular lumen, a urethral lumen, a tracheal lumen, or a nasal lumen.

21. The stent of claim 1, wherein the strip is helically arranged to be placed in a lumen having an outer diameter between about 2 mm and about 10 mm.

22. The stent of claim 1, wherein the helical strip is deformable between an active first configuration and a relaxed second configuration, such that when the strip assumes the first configuration it is biased to the second configuration.

23. The stent of claim 1, wherein the helical configuration includes a first portion having a coil with a first pitch, and a second portion having a coil with a second pitch, the second pitch being different from the first pitch.

24. The stent of claim 1, wherein the strands are bonded together.

25. A stent, comprising a generally helical body defined by a coiled strip, the coiled strip including a lumen disposed in the strip.

26. The stent of claim 25, wherein the strip includes a plurality of strands bonded together in a substantially parallel configuration.

27. The stent of claim 25, wherein a portion of the strip is biodegradable.

28. The stent of claim 25, further including a pharmaceutical agent located in the lumen.

29. The stent of claim 25, comprising a plurality of lumens.

30. The stent of claim 25, further including a carbon fiber located in the lumen.

31. The stent of claim 25, further comprising a radiopaque marker located on the strip.

32. The stent of claim 25, comprising a first biodegradable portion and a second biodegradable portion, each portion having a different degradation rate.

33. A method of making a endoluminal stent, comprising:
coupling a plurality of strands to each other in a generally parallel configuration to form a strip; and
forming the strip into a helix dimensioned to be inserted into a lumen of a patient.

34. The method of claim 33, wherein the coupling of the plurality of strands includes physically bonding one strand to another strand.

35. The method of claim 33, further comprising
causing the temperature of the strands to be between a glass transition point and a melting point;
wrapping the strands around a mandrel as the strands come off of an extruder; and
cooling the strands to cause the strands to bond to each other.

36. The method of claim 33, further comprising forming a lumen in the strip.

37. The method of claim 36, wherein the lumen is formed in a strand of the strip.

37. The method of claim 36, further comprising providing a pharmaceutical agent in the lumen of the strip.

38. The method of claim 36, further comprising providing a carbon fiber in the lumen of the strip.

39. The method of claim 35, wherein the cooling of the strands occurs before the strands are wrapped around the mandrel.

40. The method of claim 35, wherein the cooling of the strands occurs after the strands are wrapped around the mandrel.

41. The stent of claim 25, further comprising a plug located in the lumen.

42. The stent of claim 41, wherein the plug comprises a biodegradable material.

43. The stent of claim 41, comprising a plurality of plugs located in the lumen.

44. The stent of claim 43, comprising a first plug formed of a biodegradable material that degrades at a first rate, and a second plug formed of a biodegradable material that degrades at a second rate.

45. The stent of claim 41, wherein the plug is engaged with the body of the stent such that blood flow around the stent can displace the plug from the lumen when the stent is placed in a patient.

46. The stent of claim 45, wherein the blood flow pushes the plug from the lumen.

47. The stent of claim 45, wherein the blood flow pulls the plug from the lumen.

48. The stent of claim 25, wherein the strip comprises an anti-stenosis agent.

49. The stent of claim 48, wherein the strip is coated with the anti-stenosis agent.

50. The stent of claim 48, wherein the strip comprises an anti-stenosis agent disposed in the lumen of the strip.

51. The stent of claim 48, wherein the strip is formed of an anti-stenosis agent.

52. The stent of claim 25, further comprising a chemotherapeutic agent disposed in the lumen of the stent.

53. The stent of claim 1 or claim 25, wherein the stent is formed of a material and a shape effective to permit the stent to deform to different widths and shapes in a vessel.

54. The stent of claim 1 or claim 25, wherein the stent comprises a first portion having a first diameter, and a second portion having a second diameter, the second diameter being different than the first diameter.

55. The stent of claim 1, wherein the stent is configured to provide greater friction when placed in a vessel relative to a stent comprising a single strand.

56. The stent of claim 55, further comprising a projection member extending from at least one strand, the projection member being effective to engage the stent with a wall of a vessel.

57. The stent of claim 56, wherein the projection member is selected from a group consisting of cleats, ribs, spikes, and hooks.

58. The stent of claim 56, wherein the projection member is integrally formed with the at least one strand.

59. The stent of claim 55, wherein the stent includes at least one strand with an uneven surface topography.

60. The stent of claim 1 or claim 25, wherein the helical configuration is determined based on the curvature of a vessel.

61. The stent of claim 60, wherein the stent includes a left handed helical configuration.

62. The stent of claim 60, wherein the stent includes a right handed helical configuration.

63. A method of treating a patient, comprising
providing an endoluminal stent including a strand arranged in a helical configuration, and a guidewire coupled at the guidewire's distal end to the stent;
inserting the stent and guidewire into a lumen of a vessel of a patient;
uncoupling the guidewire and the stent; and
removing the guidewire from the patient.

64. The method of claim 63, wherein the endoluminal stent comprises a malleable material, and the method further comprises urging the stent into a non-helical configuration before inserting the stent into the lumen of the patient.

65. The method of claim 64, wherein the stent comprises capralactone and PLLA.

66. The method of claim 64, wherein the stent comprises a biodegradable material.

67. The method of claim 63, wherein the method comprises inserting the stent into a blood vessel in proximity to an aneurysm.

68. The method of claim 67, wherein the method comprises inserting the stent in proximity to a large mouth aneurysm.

69. The method of claim 63, wherein the guidewire is bonded to the stent.

70. The method of claim 69, wherein the guidewire is welded to the stent.

71. The method of claim 63, wherein uncoupling comprises heating the guidewire.

72. The method of claim 71, wherein the distal end of the guidewire is heated.

73. The method of claim 71, wherein the method comprises heating the guidewire while the guidewire is removed from the patient.

74. The method of claim 63, wherein the stent comprises a first stent, and the method further comprises inserting a second stent in proximity to the first stent.

75. The method of claim 74, wherein the second stent is inserted within the helix defined by the first stent.

76. The method of claim 63, wherein the stent comprises a first stent made of a biodegradable material, and the method further comprises inserting a second stent in proximity of the first stent after the first stent has degraded.

77. The method of claim 63, further comprising engaging the stent with a vessel wall of the vessel.

78. The method of claim 77, wherein the step of engaging comprises rotating the stent to cause the lumen of the vessel to enlarge.

79. The method of claim 63, wherein the stent is inserted in a contracted configuration, and when the stent is uncoupled from the guidewire, it assumes an expanded configuration.
